(19) United States
(12) Patent Application Publication
(10) Pub. No.: US 2026/0363690 A1
(43) Pub. Date: Nov. 19, 2026

(54) TISSUE-RETRACTING GUIDE FOR LOCATING IMPLANT

(71) Applicant: Zimmer, Inc., Warsaw, IN (US)

(72) Inventors: Jacob Hiday, Ann Arbor, MI (US); Jillian Gregg, Warsaw, IN (US); Kayla Guyer, Warsaw, IN (US); Gwynith Mitchell, Warsaw, IN (US); Sydney Naibauer, Warsaw, IN (US)

(21) Appl. No.: 19/673,241

(22) Filed: May 11, 2026

Related U.S. Application Data
(60) Provisional application No. 63/806,658, filed on May 15, 2025.

(51) Int. Cl. A61B 17/02; A61B 34/10; A61B 90/00

(52) U.S. Cl. CPC A61B 17/02; A61B 34/10; A61B 90/06; A61B 90/361; A61B 2034/107; A61B 2090/061

(57) ABSTRACT

A system for placing an orthopedic implant can comprise an expandable hoop retractor and a sensor module mountable to the expandable hoop retractor to obtain imaging of anatomy within the expandable hoop retractor. A method of preparing a bone for receiving a prosthetic implant can comprise producing a portal in anatomy to access a bone surface, inserting an expandable hoop retractor into the portal to displace soft tissue away from the bone surface, obtaining imaging of the bone surface from a camera attached to the expandable hoop retractor, displaying images of the bone surface on an output device, and displaying guidance indicia on the images in the output device to place a surgical instrument on the bone surface.

### CLAM OF PRIORITY

This application claims the benefit of U.S. Provisional Patent Application Serial No. 63/806,658, filed on May 15, 2025, the benefit of priority of which is claimed hereby, and which is incorporated by reference herein in its entirety.

### TECHNICAL FIELD

The present disclosure is generally directed to, but not by way of limitation, systems, devices and methods for performing orthopedic procedures, such as those related to implanting prosthetic devices into bone. More specifically, but not by way of limitation, the present disclosure is directed to systems, devices and methods for accessing bone and determining locations on the bone for placement of a prosthetic component.

### BACKGROUND

A shoulder joint or glenohumeral joint includes a generally ball-shaped head of the humerus that articulates with the glenoid or socket of the scapula. In a healthy shoulder joint, articular cartilage covers the articular portions of the humeral head and the glenoid to facilitate movement of the shoulder joint. However, due to disease or traumatic injury, for example, the articular cartilage of the shoulder joint may become damaged or degenerated.

Such changes to the shoulder anatomy can result in the desire to replace of all or part of the natural shoulder joint with one or more prosthetic shoulder components. For example, the natural humeral head can be replaced with a prosthetic humeral component. Also, the glenoid can be replaced with a prosthetic glenoid component. In a total shoulder arthroplasty, the glenoid is typically resurfaced and shaped to accept a prosthetic glenoid component and the humeral head is resected to accept a prosthetic humeral head component. However, partial arthroplasty procedures can be performed where only one of the glenoid and humeral head are replaced. In reverse shoulder procedures, the glenoid is typically resurfaced and shaped to accept a prosthetic humeral head component and the humeral head is resected to accept a prosthetic glenoid component in another version of a total shoulder arthroplasty procedure.

In typical shoulder arthroplasty procedures, as well as arthroplasty procedures at other anatomic locations, e.g., hip joints and knee joints, the joint is accessed through soft tissue at the anatomic location. For example, in shoulder arthroplasty procedures, the glenoid can be accessed by opening the rotator cuff or via inferior glenohumeral release to improve range of motion and access the glenoid. Any of the glenohumeral ligaments (superior, middle, and inferior), the subscapularis muscle, and the coracohumeral ligament can obstruct access to the arthroplasty location, thereby making it desirable to retract one or more of these tissues. Thus, gaining access to the glenoid during a total shoulder replacement procedure can be challenging. Various types of retractors exist to hold soft tissue in a retracted state to allow access to the glenoid. In some examples, the retractors are held in place by surgical personnel during the surgical procedure, thereby increasing the number of people for performing the procedure or preventing personnel from performing more skilled tasks.

Examples of retractors are described in: Pub. No. US 2017/0079803 A1 to Lang, titled “Methods, Devices and Techniques for improved Placement and Fixation of Shoulder Implant Components”; Pat. No. US 9,107,629 B2 to Anthony et al., titled “Spreading Retractor”; and Pat. No. US 11,607,320 B2 to Greiwe, titled “Humeral Head Implant System.”

### OVERVIEW

The present inventors have recognized, among other things, that problems to be solved in accessing bone locations within anatomic joints involve retracting and holding soft tissue away from the location on the bone where an orthopedic procedure is to be performed. As is known, various retractors can be used to pull and hold soft tissue away from an anatomic site. However, conventional retractors have various shortcomings. For example, some retractors can hold back tissue, but adjacent tissue can remain un-retracted. Additionally, it can be common for some tissue to slip off the retractor and return to an un-retracted position over the location being accessed. Additionally, some retractors can themselves obstruct visibility of the desired anatomic location.

Another problem associated with arthroplasty procedures is locating anatomic landmarks on the bone once accessed at the anatomic location. Even with soft tissue retracted or cleared away, it can be difficult to visualize or decipher anatomic contours on the surface of the bone due to the presence of blood, lighting conditions and the diseased state of the bone. As such, it can be difficult for a surgeon to determine where to perform desired bone modifications to accept the prosthetic component.

The present subject matter can provide solutions to these and other problems, such as by providing a tissue-locating guide system for arthroplasty procedures that can securely retract soft tissue away from a location on a bone surface where an arthroplasty procedure is to be performed. The retractor can comprise a hoop or ring that can be placed in engagement with bone structure to restrain soft tissue around a perimeter of the arthroplasty location. The retractor can be adjustable in size, e.g., perimeter, to allow the retractor to be constricted for insertion into a soft tissue portal and then expanded to a specific size of a particular glenoid to expose more surface area of the bone. The tissue-locating guide system for arthroplasty procedures can comprise an imaging system that can obtain images from an imaging device mounted to a retractor, such as one of the aforementioned expandable hoop retractors. The retractor can provide a stable platform for coupling to the imaging device. The imaging system can generate output, such as video images, of the bone structure. The imaging system can analyze contours of bone structure in the obtained images and identify bone landmarks useful in performing an arthroplasty procedure. The imaging system can overlay indicia or other guidance information or text onto the bone landmarks that provide guidance to a surgeon as to where to place instrumentation for performing operations on the bone structure, such as placement to pins that facilitate reaming, drilling and the like. As such, the surgeon can view real-time or live images of a bone surface with soft tissue retracted so that an instrument can be viewed in relation to the guidance indicia to move, e.g., guide, the instrument into a desired to location to perform a step in a surgical procedure, e.g., placement of a pin.

In an example, a system for placing an orthopedic implant can comprise an expandable hoop retractor and a sensor module mountable to the expandable hoop retractor to obtain imaging of anatomy within the expandable hoop retractor.

In another example, a method of preparing a bone for receiving a prosthetic implant can comprise producing a portal in anatomy to access a bone surface, inserting an expandable hoop retractor into the portal to displace soft tissue away from the bone surface, obtaining imaging of the bone surface from a camera attached to the expandable hoop retractor, displaying images of the bone surface on an output device, and displaying guidance indicia on the images in the output device to place a surgical instrument on the bone surface.

### BRIEF DESCRIPTION OF THE DRAWINGS

FIG. 1 is a perspective view of a tissue-locating guide system of the present disclosure comprising a retractor connected to a camera assembly.

FIG. 2A is a perspective view of the retractor of FIG. 1 comprising an expandable hoop retractor.

FIG. 2B is an exploded view of the expandable hoop retractor of FIG. 2A showing an arcuate body, a first hinge, a second hinge, a latch and a plurality of pins.

FIG. 3A is a side view of a stopper pin that can be inserted into the expandable hoop retractor of FIG. 2A to prevent constriction thereof.

FIG. 3B is a front view of the stopper pin of FIG. 3A showing connection to a coupler of the camera assembly.

FIG. 4 is a perspective view of the camera assembly of FIG. 1 comprising a sensor module, an attachment neck and a coupler.

FIG. 5 is an end view of the camera module of FIG. 4 showing an imaging sensor encased within a transparent housing.

FIG. 6 is a side view of the attachment neck of FIG. 4 showing a first end for connecting to the stopper pin and a second end for connecting to the camera module.

FIG. 7 is a schematic view of the tissue-locating guide system of FIG. 1 through FIG. 6 connected to a computer system and output device.

FIG. 8 is an example of output of the computer system of FIG. 7 showing a contour map of a glenoid.

FIG. 9 is an example of guidance indica generated by the computer system of FIG. 7 displayed over the contour map of the glenoid of FIG. 8.

FIG. 10 is a block diagram illustrating operations of various methods of performing an arthroplasty procedure using the tissue-locating guide systems of the present disclosure.

FIG. 11 is a block diagram of an example machine upon which any one or more of the techniques and methods discussed herein can be performed and with which any of the devices discussed herein can be used in accordance with some embodiments.

FIG. 12A is a schematic plan view of an example of an expandable hoop retractor of the present disclosure comprising a plurality of linkages pivotably linked together in a collapsed state.

FIG. 12B is a schematic plan view of the expandable hoop retractor of FIG. 12A in an expanded state.

### DETAILED DESCRIPTION

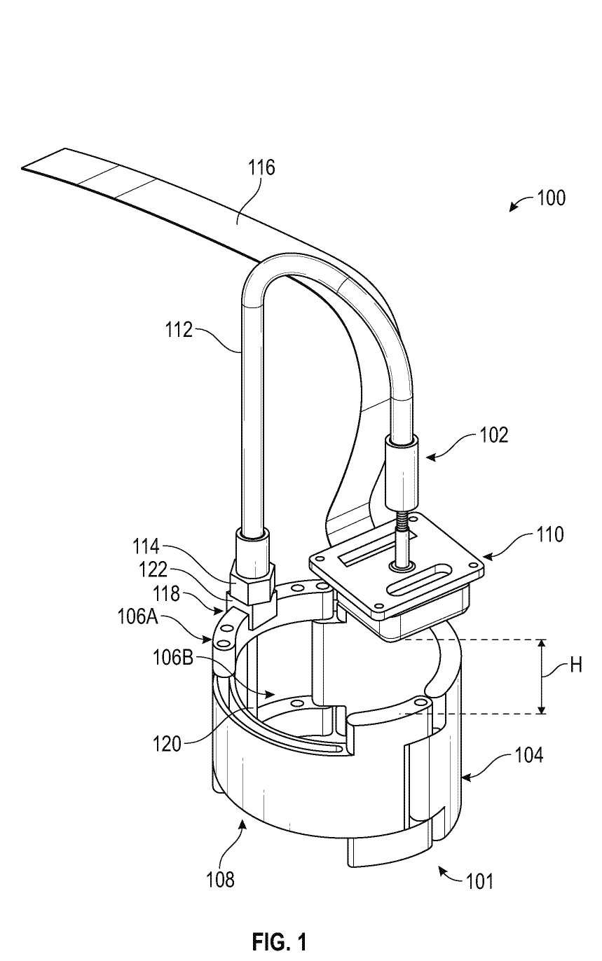

FIG. 1 is a perspective view of tissue-locating guide system 100 of the present disclosure comprising retractor 101 connected to camera assembly 102. Retractor 101 can comprise arcuate body 104, first hinge 106A, second hinge 106B and latch 108. Camera assembly 102 can comprise sensor module 110, attachment neck 112, retractor coupler 114 and wiring 116. Stopper pin 118 can be inserted into retractor 101 to control a size of retractor 101 and to facilitate coupling to camera assembly 102. Stopper pin 118 can comprise pin 120 and head 122.

Retractor 101 can comprise an annular or closed-loop body, such as a hoop or ring. During use, arcuate body 104, first hinge 106A, second hinge 106B and latch 108 can be seated on or in close proximity to a bone surface. Arcuate body 104, first hinge 106A, second hinge 106B and latch 108 can prevent and/or inhibit soft tissue, such as skin, muscle, tendon and the like, from encroaching on the bone surface within retractor 101. As discussed herein, retractor 101 can have an expandable and contractable diameter or perimeter. Stopper pin 118 can be inserted into retractor 101 to limit the amount that retractor 101 can be expanded and/or contracted.

During use, camera assembly 102 can be mounted to retractor 101 to facilitate capture of imaging of the bone surface within retractor 101. In examples, camera assembly 102 can be mounted to stopper pin 118. In examples, camera assembly 102 can be mounted directly to retractor 101. Retractor 101 can provide a stable platform for camera assembly 102 such that consistent and steady imaging can be obtained. For example, engagement of retractor 101 with the bone surface can prevent jostling or shaking of camera assembly 102. The hoop-like shape of retractor 101 can allow for attachment of camera assembly 102 to retractor 101 in a desired location relative to the anatomy. The hoop-like shape of retractor 101 can allow for retractor to be rotated while seated in the soft tissue of the anatomy to facilitate positioning of camera assembly 102 and to better obstruct soft tissue.

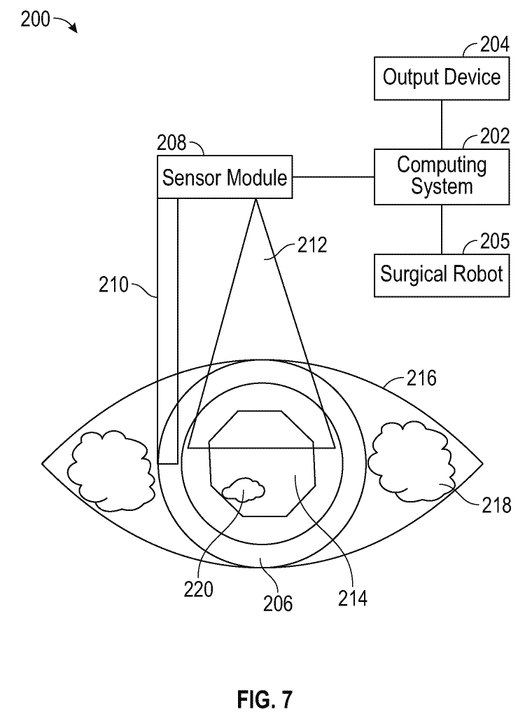

During use, camera assembly 102 can be used to obtain imaging of bone structure within retractor 101. Retractor coupler 114 can comprise a body to attach to stopper pin 118. Attachment neck 112 can extend from retractor coupler 114 to position sensor module 110 within or partially within a cylinder defined by the perimeter of retractor 101. In examples, attachment neck 112 can be rigid to position sensor module 110 in a fixed location. In examples, attachment neck 112 can be flexible to allow for variable positioning of sensor module 110. Sensor module 110 can comprise one or more imaging devices to obtain images of anatomy proximate retractor 101. As discussed herein, sensor module 110 can additionally include other devices for obtaining feedback from anatomy proximate retractor 101, such as lidar or other distance sensors that can be used to generate three-dimensional depth information and indicia. Sensor module 110 can be connected to a computing system, e.g., computing system 202 of FIG. 7, via wiring 116. Sensor module 110 can obtain real-time feedback from the anatomy to allow the computing system to generate images and guidance indicia that can allow a surgeon or other personnel to view live video of the anatomy to place instrumentation relative to the anatomy, as discussed in greater detail with reference to FIG. 7 through FIG. 9.

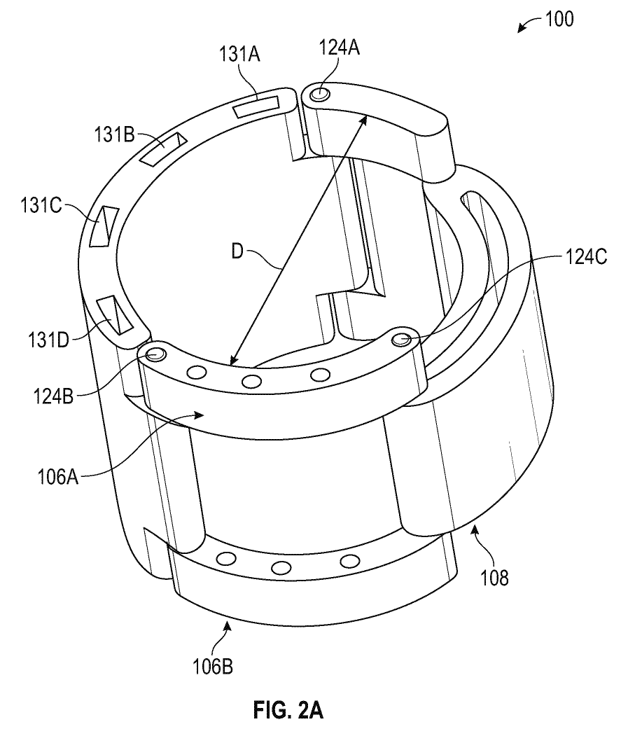

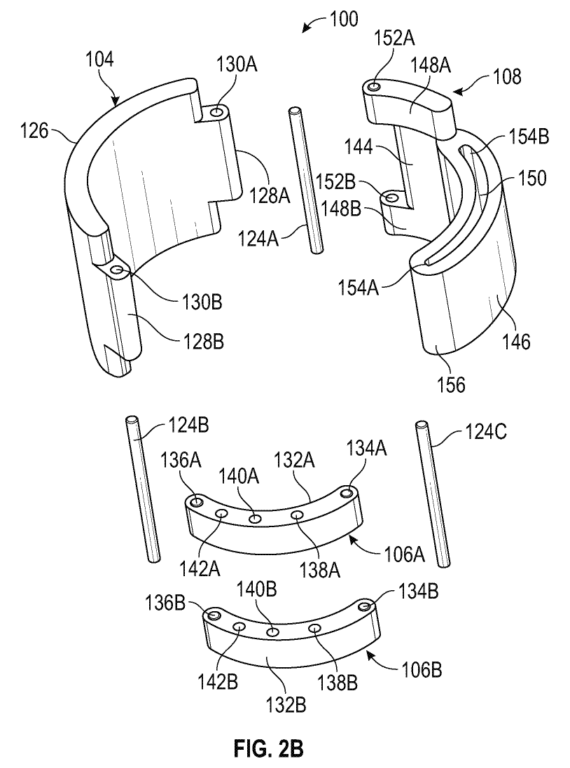

FIG. 2A is a perspective view of retractor 101 of FIG. 1 comprising a plurality of components that can be linked together to form an annular body having a variable perimeter size and/or shape. FIG. 2B is an exploded view of retractor 101 of FIG. 2A showing arcuate body 104, first hinge 106A, second hinge 106B and latch 108. Retractor 101 can further comprise first pin 124A, second pin 124B and third pin 124C. FIGS. 2A and 2B are discussed concurrently.

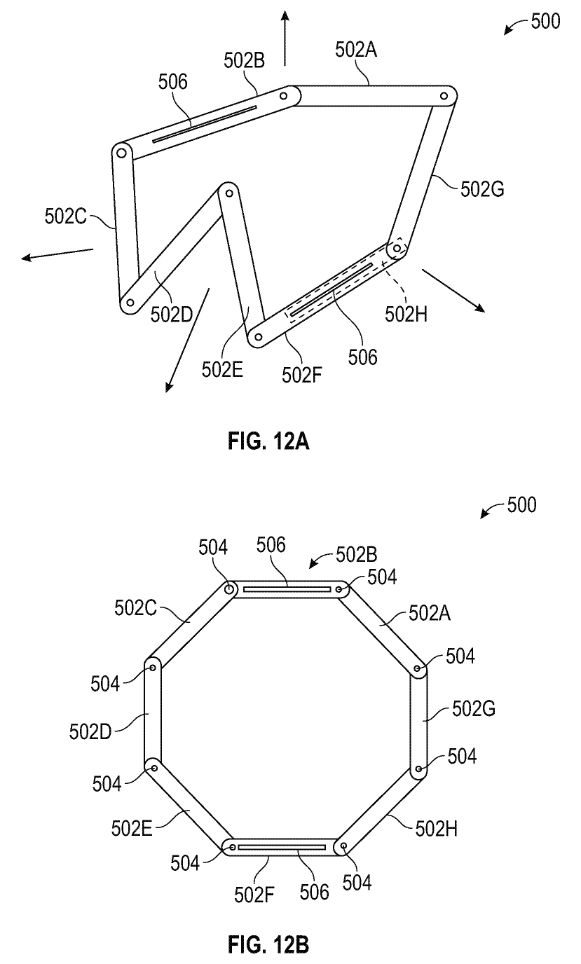

In the illustrated example, the components of retractor 101 can be linked to form a circular shape. However, retractor 101 can have other shapes, such as oval, elliptical, racetrack, octagonal, hexagonal, rectangular, and square. FIGS. 12A and 12B illustrate another example of an expandable hoop retractor comprising a plurality of pivoting linkages with eight sides. The components of retractor 101 can be fabricated from any suitable material, such as biocompatible materials including plastics, polymers and metals.

Arcuate body 104 can comprise wall 126, first flange 128A and second flange 128B. First flange 128A can comprise first bore 130A and second flange 128B can comprise second bore 130B. Wall 126 can comprise a solid component. However, in examples, wall 126 can include slot 131A, slot 131B, slot 131C and slot 131D that can be used to connect to camera assembly 102, for example. In examples, one or more pins can be placed into and partially through one or more of slot 131A through slot 131D, or any other bore or hole in retractor 101, to extend into bone matter to anchor, affix or secure retractor 101 in place. Wall 126 can be semi-circular in shape or can comprise a smaller segment of a circle. However, wall 126 can have other shapes, such as other curved shapes that are not circular. First flange 128A and second flange 128B can comprise extensions of wall 126 that extend along the trajectory of the curvature of wall 126 or can extend from wall 126 at another trajectory or angle. First flange 128A and second flange 128B can extend from wall 126 to provide structure for attaching to first hinge 106A, second hinge 106B and latch 108. First bore 130A of first flange 128A and second bore 130B of second flange 128B can be configured to receive first pin 124A and second pin 124B, respectively. First bore 130A and second bore 130B can comprise smooth or through bores. However, first bore 130A and second bore 130B can be threaded.

First hinge 106A can comprise first member 132A, first hinge bore 134A, second hinge bore 136A, and a plurality of adjuster holes. Examples first adjuster hole 138A, second adjuster hole 140A and third adjuster hole 142A.

Second hinge 106B can comprise second member 132B, first hinge bore 134B, second hinge bore 136B, and a plurality of adjuster holes. Examples first adjuster hole 138B, second adjuster hole 140B and third adjuster hole 142B.

First member 132A and second member 132B can comprise bodies that can be pivotably linked to second flange 128B via second pin 124B at first ends and that can be pivotably linked to latch 108 at second ends. First member 132A and second member 132B can comprise solid bodies. However, first member 132A and second member 132B can include first hinge bore 134A through third adjuster hole 142B, respectively. Second pin 124B can be inserted through second hinge bore 136A, second bore 130B and second hinge bore 136B to couple first member 132A and second member 132B to arcuate body 104. Second pin 124B can be configured to remain in contact with arcuate body 104, first member 132A and second member 132B when inserted therein. In examples, second pin 124B can be attached via an interference fit. In examples, second pin 124B can be attached via threaded engagement. In examples, fasteners, such as nuts, can be attached to second pin 124B.

Latch 108 can comprise wall 144, slide body 146, first flange 148A, second flange 148B and slot 150.

First flange 148A and second flange 148B can comprise bodies that can be pivotably linked to first flange 128A via first pin 124A at first ends. First flange 148A and second flange 148B can comprise solid bodies. However, first flange 148A and second flange 148B can include first hinge bore 152A and second hinge bore 152B, respectively. First pin 124A can be inserted through first hinge bore 152A, first bore 130A and second hinge bore 152B to couple latch 108 to arcuate body 104. First pin 124A can be configured to remain in contact with arcuate body 104, first flange 148A and second flange 148B when inserted therein. In examples, first pin 124A can be attached via an interference fit. In examples, first pin 124A can be attached via threaded engagement. In examples, fasteners, such as nuts, can be attached to first pin 124A.

Slide body 146 can extend from wall 144 to extend toward first hinge 106A and second hinge 106B. Slide body 146 can fit between first hinge 106A and second hinge 106B when first hinge 106A and second hinge 106B are attached to arcuate body 104. The combined height of slide body 146, first flange 128A and second flange 128B can be approximately equal to the height of wall 126 and the height of wall 144.

Third pin 124C can be inserted into slot 150, first hinge bore 134A and first hinge bore 134B to control the outer perimeter size of retractor 101. In particular, third pin 124C can be used to control the magnitude of diameter D (FIG. 2A). Thus, third pin 124C can be permitted to slide all the way from first end 154A of slot 150 to second end 154B of slot 150. In examples the length of slide body 146 can be sufficiently long to allow the lengths of first hinge 106A and second hinge 106B that extend beyond second flange 128B to overlap with slide body 146. Thus, the maximum size of retractor 101, e.g., the maximum diameter D, can be when third pin 124C engages first end 154A of slot 150 and the minimum size of retractor 101, e.g., the minimum diameter D, can be when third pin 124C engages second end 154B of slot 150.

In practice, when retractor 101 is inserted into anatomy, soft tissue surrounding retractor 101 can generate radially inward forces that tend to collapse retractor 101 such that third pin 124C has a tendency to slide toward second end 154B of slot 150. Thus, in order to prevent retractor 101 from collapsing to its smallest diameter, another pin can be inserted into one of first adjuster hole 138A, second adjuster hole 140A and third adjuster hole 142A, as well as one of first adjuster hole 138B, second adjuster hole 140B and third adjuster hole 142B, outside of slot 150 to limit the ability of slide body 146 to be inserted between first hinge 106A and second hinge 106B. Such a pin can thus resist the radial inward forces of the soft tissue by engaging end wall 156 of slide body 146. In examples, the pin for engaging first adjuster hole 138A, second adjuster hole 140A and third adjuster hole 142A, as well as first adjuster hole 138B, second adjuster hole 140B and third adjuster hole 142B, can comprise pin 120 of stopper pin 118 (FIG. 1).

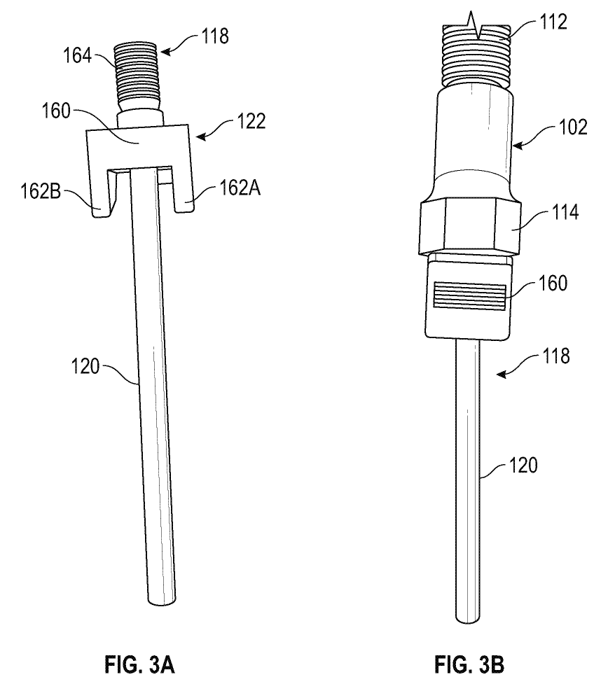

FIG. 3A is a side view of stopper pin 118 that can be inserted into retractor 101 of FIG. 2A to prevent constriction thereof. FIG. 3B is a front view of stopper pin 118 of FIG. 3A showing connection to retractor coupler 114 of camera assembly 102. FIGS. 3A and 3B are discussed concurrently.

Stopper pin 118 can comprise pin 120 and head 122. Head 122 can comprise block 160, first flange 162A, second flange 162B and fastener 164. Block 160 can comprise a body configured to attach to pin 120. In examples, block 160 can comprise a bore for receiving pin 120. In examples, pin 120 can be threadedly engaged with block 160. In examples, pin 120 can be configured to be removed from block 160 so that block 160 can be attached to retractor 101 separately from pin 120, such as via insertion of one of first flange 162A or second flange 162B into one of slot 131A through slot 131D. First flange 162A and second flange 162B can project from block 160 in the direction of pin 120. In examples, first flange 162A and second flange 162B can extend parallel to pin 120. First flange 162A and second flange 162B can be positioned to straddle first hinge 106A to allow pin 120 to be inserted into bores therein when block 160 engages the top or proximal surface of first hinge 106A. In examples, first flange 162A and second flange 162B can be configured to pinch or squeeze first hinge 106A to prevent dislodgment of block 160 from first hinge 106A. Fastener 164 can extend proximally from block 160. Fastener 164 can comprise a feature, body or projection configured to engage with retractor coupler 114 of camera assembly 102. In examples, fastener 164 can comprise a threaded post configured to be engaged with mating threading in a socket or bore inside retractor coupler 114. Stopper pin 118 can be sized for insertion into first adjuster hole 138A, second adjuster hole 140A and third adjuster hole 142A, as well as first adjuster hole 138B, second adjuster hole 140B and third adjuster hole 142B. In examples, stopper pin 118 can be sized to produce an interference fit with the bores of first hinge 106A and second hinge 106B.

Retractor 101 and stopper pin 118 can work together to retract soft tissue from an anatomic location. In example, stopper pin 118 can be removed from retractor 101 to allow retractor 101 to be collapsed or constricted to a smaller outer perimeter size. Thereafter, the collapsed retractor 101 can be inserted into an access portal in soft tissue. The collapsed retractor 101 can be pushed down to engage a bone surface. Subsequently, retractor 101 can be expanded to push the soft tissue further away from the anatomic location, and stopper pin 118 can be inserted therein to prevent retractor 101 from collapsing down to a smaller size.

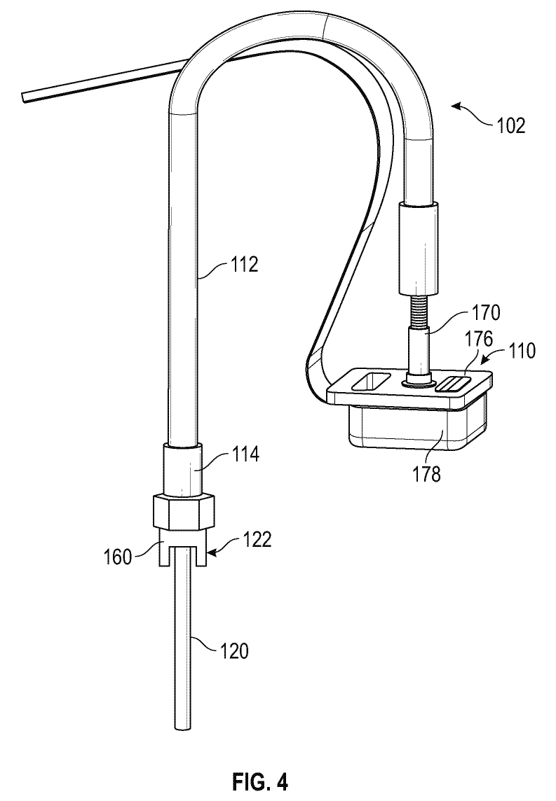

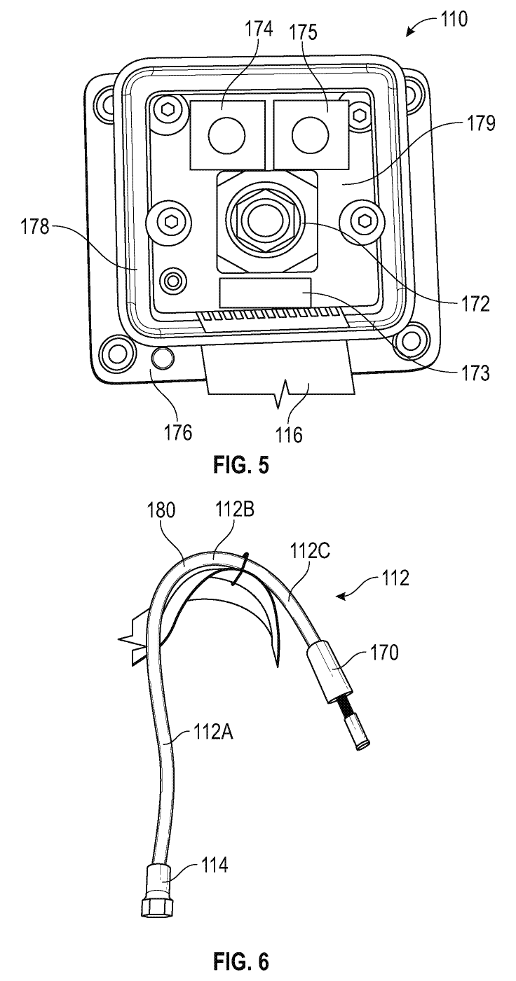

FIG. 4 is a perspective view of camera assembly 102 of FIG. 1 comprising sensor module 110, attachment neck 112, retractor coupler 114 and camera coupler 170. FIG. 5 is an end view of sensor module 110 of FIG. 4 showing imaging sensor 172 and distance sensor 174 attached to sensor board 176. Sensor board 176 can be coupled to housing 178 to protect imaging sensor 172 and distance sensor 174. FIG. 6 is a side view of attachment neck 112 of FIG. 4 showing a first end having retractor coupler 114 for connecting to stopper pin 118, a second end having camera coupler 170 for connecting to sensor module 110 and articulating section 180 therebetween. FIG. 4, FIGS. 5 and 6 are discussed concurrently.

Articulating section 180 can be configured to position sensor module 110 above or superior of retractor 101. In examples, articulating section 180 can position sensor module 110 a distance or height H (FIG. 1) above or away from retractor 101 to produce clearance therebetween. As such, instrumentation, medical devices and the like can be inserted into retractor 101 to engage with anatomy, e.g., a bone surface, therein with reduced interference from camera assembly 102. In examples, the shape of articulating section 180 can be configured to place sensor module 110 in a relative position relative to retractor 101, such as to be able to obtain imaging from within retractor 101. In examples, retractor 101, articulating section 180 and sensor module 110 can be configured to obtain images at the center of retractor 101. In examples, articulating section 180 and sensor module 110 can be configured to obtain images of the entirety of the interior of retractor 101, such as a single image that covers all of retractor 101.

Articulating section 180 can comprise a flexible portion of attachment neck 112 that is bendable so as to be deflectable when subject to an articulation force, such as input from a user. Articulating section 180 can be configured to hold its position when the articulation force is removed. In examples, articulating section 180 can be fabricated from a gooseneck tube, such as those fabricated from a round coil spring interlaced with a wedge-shaped coil. In examples, articulating section 180 can have a fixed or rigid shape that is not intended to be changed by a user during typical usage. In examples, articulating section 180 has a U-shape having first leg 112A connected to retractor coupler 114, second leg 112B and third leg 112C connected to camera coupler 170, wherein first leg 112A is straight, second leg 112B is curved or semi-circular and third leg 112C is straight and shorter than first leg 112A. Articulating section 180 can have different shapes in different examples.

Retractor coupler 114 and camera coupler 170 can comprise components of fasteners configured to attach articulating section 180 to retractor 101 and sensor module 110, respectively. In examples, retractor coupler 114 and camera coupler 170 can be the same type of component or different types of components. In examples, retractor coupler 114 can comprise a threaded socket for receiving a threaded post of head 122 and camera coupler 170 can comprise a threaded socket for receiving a threaded post extending from a rear or back side of sensor board 176.

Sensor board 176 can comprise a platform or structure for mounting of electronics components of sensor module 110. In examples, circuit board 179 can be mounted to a front or forwards side of sensor board 176. Circuit board 179 can be configured to electronically couple electrical components of sensor module 110, such as imaging sensor 172 and distance sensor 174, as well as other components described herein such as a wireless communication device.

In examples, imaging sensor 172 can comprise a device, sensor or imager, that is configured to detect and convey information to form an image by converting light or other electromagnetic radiation into electrical signals, which are then processed to create a digital image. In examples, imaging sensor 172 can comprise a complementary metal oxide semiconductor (CMOS) sensor, a charge-coupled device (“CCD” sensor) or the like. In examples, imaging sensor 172 can comprise a Chip-on-the-Tip image sensor, such as those used in endoscopes.

In examples, distance sensor 174 can be configured to generate one or more signals indicative of the distance of surfaces or objects from distance sensor 174. In examples, distance sensor 174 can comprise an ultrasonic sensor, an infrared (IR) proximity sensor, a laser distance sensor, a light detection and ranging sensor (LIDAR) and the like.

In examples, sensor module 110 can include light generator 175 mounted to circuit board 179. In examples, sensor module 110 can comprise a light emitting diode (LED) or the like. In examples, light generator 175 can comprise a laser that is configured to emit instructions or indicia for performing a surgical procedure, as discussed herein, from computing system 202 directly onto a bone surface.

In examples, sensor module 110 can include wireless communication device 173 mounted to circuit board 179 that is configured to communicate via wireless communication signals, such as Bluetooth, WiFi, Zigbee, infrared (IR), near field communication (NFC), 3GPP or other technologies. As illustrated, sensor module 110 can include wiring 116 to electronically connect circuit board 179 and electronic components connected thereto to an external computing device, such as computing system 202 of FIG. 7.

Housing 178 can be positioned over circuit board 179 and the components mounted thereto to protect circuit board 179. Housing 178 can comprise a bubble or blister configured to cover or encapsulate circuit board 179. In examples, housing 178 can be attached to circuit board 179 in a sealed manner such that fluids are prevented or inhibited from entering between sensor board 176 and housing 178. In examples, housing 178 can be fabricated from a hard or rigid material to prevent damage to circuit board 179 and components attached thereto. In examples, housing 178 can be fabricated from a clear or transparent material, such as transparent polycarbonate, to allow light to pass through housing 178.

As such, camera assembly 102 can be configured to obtain data from anatomy disposed within or below retractor 101. The imaging can be used to generate two-dimensional and three-dimensional imaging data representing such anatomy. The imaging data can be analyzed by a computing system to determine landmarks and features of the anatomy. Furthermore, the computing system can be used to generate guidance indicia or instructions that can be overlayed over the imaging date to facilitate a surgeon or other personnel positioning of surgical hardware at desired locations in the anatomy.

FIG. 7 is a schematic view of tissue-locating guide system 200 connected to computing system 202, output device 204 and surgical robot 205. In examples, tissue-locating guide system 200 can comprise the tissue-locating guide system of FIGS. 1-6.Tissue-locating guide system 200 can additionally comprise retractor 206 and sensor module 208. Retractor 206 can comprise retractor 101 of FIGS. 1-2B or retractor 500 of FIGS. 12A and 12B. Sensor module 208 can comprise sensor module 110 of FIG. 1, FIGS. 4 and 5. Sensor module 208 can be connected to retractor 206 via extension 210, which can comprise attachment neck 112. Sensor module 208 can be configured to have field of view 212, which can additionally comprise a signal emission onto target location 214.

Retractor 206 can be positioned within portal 216 made in an anatomic structure, such as a shoulder of a patient. However, tissue-locating guide system 200 can be configured for use with other anatomic locations, such as knee, hip, ankle, spine and the like. Portal 216 can comprise an incision through the skin to access target location 214. In examples, target location 214 can comprise a bone surface, such as a glenoid. Retractor 206 can be positioned within portal 216 to contact bone of or around target location 214. Retractor 206 can be configured to prevent soft tissue 218 within portal 216 from extending over or onto target location 214. Soft tissue 218 can comprise muscle, fat, ligaments and the like. Extension 210 can be connected to retractor 206 to position sensor module 208 proximate, e.g., over target location 214. Sensor module 208 can have field of view 212 to obtain images of target location 214. Additionally, sensor module 208 can emit a signal coincident with field of view 212 configured to obtain distance readings to contours or shapes of target location 214. Field of view 212 can additionally include or comprise a light signal to illuminate target location 214. Sensor module 208 can generate images representing target location 214 and send such signals to computing system 202. Likewise, distance information from sensor module 208 can be sent to computing system 202. Computing system 202 can be in wired communication with sensor module 208, such as via wiring 116, or can be in wireless communication with sensor module 208, such as via light generator 175. Output device 204 can comprise a device for displaying digital images or video, such as a television, computer monitor, LCD screen, LED screen or the like. In examples, output device 204 can additionally be configured to receive user inputs, such as via a mouse, keyboard or touchscreen. In example, output device 204 can comprise a computer, mobile phone, tablet computer or the like. Computing system 202 can be configured to process signals from sensor module 208 to generate output for display on output device 204. For example, computing system 202 can be configured to generate still images or video images of target location 214. Target location 214 can include diseased bone matter 220. Computing system 202 can also be configured to generate guidance indicia over the digital images of the anatomy to facilitate performance of a surgical procedure.

In additional examples of the present disclosure, instructions and indicia generated for placing surgical components on or at target location 214 can be converted to instructions for operating surgical robot 205. In examples, the instructions can comprise three-dimensional coordinates in a tracking system for placing the surgical component at target location 214. In examples, tissue-locating guide system 200 can comprise a 3D tracking system, such as an optical tracking system, that can determine the location of objects attached to trackers, e.g., reflectors, including those attached to retractor 206, sensor module 208 and surgical robot 205. Thus, an articulating arm of surgical robot 205 and controlled by computing system 202 can place the surgical component at the desired location without user input.

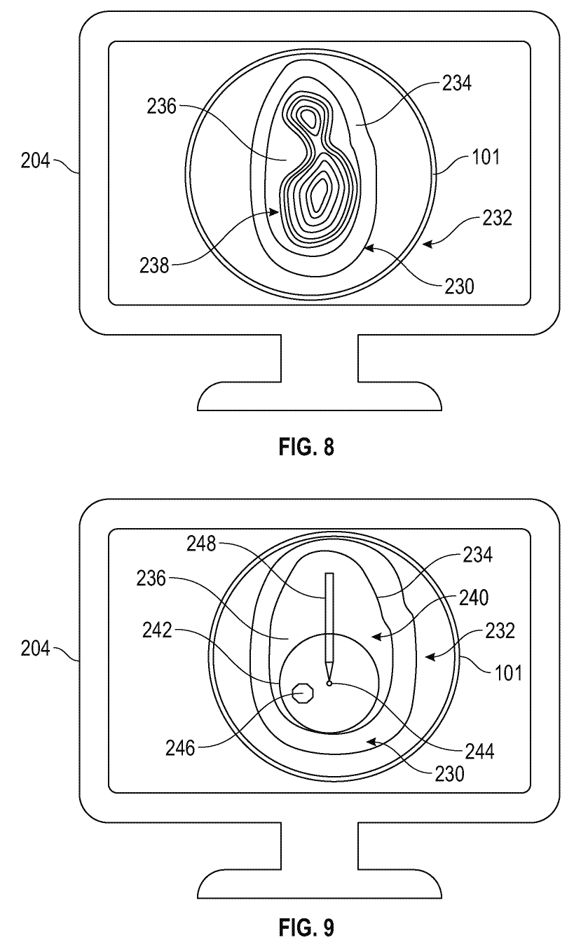

FIG. 8 is an example of output of computing system 202 of FIG. 7 showing contour map 230 of glenoid 232 on output device 204. Contour map 230 can comprise first outline 234 of anatomic feature 236. In examples, first outline 234 can comprise a labrum and anatomic feature 236 can comprise a glenoid cavity of glenoid 232. Contour map 230 can provide highly distinct indicia of the features of glenoid 232 to allow a person, such as a surgeon or surgical technician, to easily and readily recognize anatomic features. Thus, first outline 234 can comprise a brightly colored band outlining the labrum. Anatomic feature 236 can be filled-in or overlayed with other indicia to facilitate recognition of features, e.g., protrusions and/or depressions thereon. In examples, anatomic feature 236 can have contour lines 238 disposed therein. Contour lines 238 can comprise indicia configured to facilitate determination, recognition and display of the different depth levels of glenoid 232. As discussed herein, first outline 234, anatomic feature 236 and contour lines 238 can be derived from imaging of glenoid 232, such as from images obtained from imaging sensor 172 of sensor module 110 (FIG. 5). In examples, computing system 202 (FIG. 7) can be configured to run image recognition algorithms and/or software to determine the presence and location of features within anatomic structures. In examples, multiple images from imaging sensor 172 can be analyzed, such as from multiple angles can be analyzed to determine three-dimensional depth of anatomic feature 236. Additionally, or alternatively, distance sensor 174 can additionally be used to determine three-dimensional depth of anatomic feature 236. Three-dimensional depth determinations or measurements of anatomic feature 236 can be used to generate and display contour lines 238. Thus, computing system 202 can generate raw video and enhanced video of anatomy to help a user visualize anatomic features relevant to a particular procedure, which can then be further interpreted by computing system 202 to generate guidance indica for instrument placement to perform the particular procedure.

FIG. 9 is an example of guidance indica 240 generated by computing system 202 of FIG. 7 displayed over contour map 230 of glenoid 232 of FIG. 8. Guidance indica 240 can comprise work area 242, target 244 and no-go zone 246. Furthermore, output device 204 can show instrument 248. In examples, instrument 248 can comprise a pin or a marking device, such as a marker, a pen or a Bovie, e.g., an electrosurgical cutting device. Work area 242 can delineate an area on anatomic feature 236 where it is generally desirable to place an instrument or implant. In the illustrated example, work area 242 comprises a circular boundary. However, other shaped boundaries can be displayed, such as square, rectangular, octagonal, hexagonal or other non-geometric or irregular shapes. Target 244 can comprise precise placement indicia for where to place instrument 248. Thus, target 244 can comprise a dot, cross, X or crosshair for where to place instrument 248. In examples, placement of target 244 can be at the center or central position within work area 242. However, in examples, placement of target 244 within work area 242 can be adjusted based on the presence of no-go zone 246. In examples, no-go zone 246 can comprise diseased or compromised bone structure of anatomic feature 236. No-go zone 246 can be recognized in images from sensor module 110 by computing system 202, such as by using machine learning or image recognition algorithms or software. A surgeon, or another person, or a robotic arm can move instrument 248 within the field of view of sensor module 110 to display instrument 248 on output device 204. Thus, guidance indica 240 can assist a surgeon in placing instrument 248 on actual anatomy, rather than the surgeon having to rely exclusively on manual, non-augmented visualization of the anatomy with their own eyes. Alternatively, as mentioned an articulating arm of surgical robot 205 can place instrument 248 via movement to a 3D coordinate location for target 244 without user intervention.

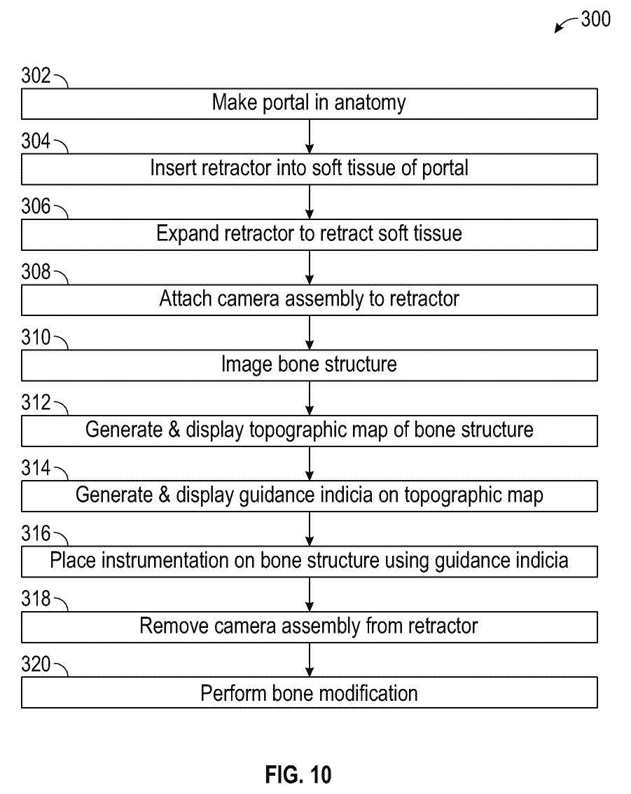

FIG. 10 is a block diagram illustrating operations of various methods 300 of performing a surgical procedure, such as an arthroplasty procedure, using the tissue-locating guide systems, e.g., tissue-locating guide system 100, of the present disclosure. FIG. 10 is discussed with reference to tissue-locating guide system 100 of FIGS. 1-9, but operation 302 through operation 320 can be used with any retractor and guidance system consistent with the present disclosure. In examples, some of operation 302 through operation 320 can be omitted and in examples operation 302 through operation 320 can be performed in other sequences. Operation 302 through operation 320 are discussed with reference to performing a shoulder arthroplasty, but can be used in performing other arthroplasties and other surgical procedures using retractors in open portals.

At operation 302, a portal can be made within anatomy. For example, an incision can be made in skin to cut through one or more of the epidermis, fat layers, muscle, tendons ligaments and the like to reach a bone structure. For example, a scalpel can be used to cut through soft tissue. In examples, portal 216 (FIG. 7) can be produced in a shoulder to access a glenoid.

At operation 304, a retractor can be inserted into soft tissue of the portal. In examples, retractor 101 can be inserted into the portal made at operation 302. Retractor 101 can be collapsed, compacted or restricted to a smaller size or diameter to better fit into portal 216. Retractor 101 can be advanced or pushed down into portal 216 to come into close proximity to or contact with a bone structure, such as a glenoid. In examples, retractor 500 of FIGS. 12A and 12B can be used. In examples, one or more pins can be placed into and partially through one or more of slot 131A through slot 131D and first adjuster hole 138A, second adjuster hole 140A and third adjuster hole 142A, as well as one of first adjuster hole 138B, second adjuster hole 140B and third adjuster hole 142B to extend into bone matter to anchor, affix or secure retractor 101 in place.

At operation 306, the retractor can be expanded to retract soft tissue. Retractor 101 can be expanded, widened or enlarged to push soft tissue further away from the bone structure. As such, retractor 101 can restrain soft tissue from impinging upon or extending over the bone structure. In examples, retractor 101 can be locked or restrained from changing back to the collapsed or compacted configuration by forces of the soft tissue. For example, pin 120 (FIG. 3A) can be inserted into one of first adjuster hole 138A, second adjuster hole 140A and third adjuster hole 142A, as well as first adjuster hole 138B, second adjuster hole 140B and third adjuster hole 142B, to prevent retractor 101 from collapsing.

At operation 308, a camera module can be attached to the retractor. For example, sensor module 110 can be attached to retractor 101. For example, retractor coupler 114 can be attached to pin 120 or head 122 can be attached directly to retractor 101. Sensor module 110 can be positioned relative to retractor 101 to obtain images of the bone structure within retractor 101.

At operation 310, images of a bone structure within the retractor can be obtained. Imaging sensor 172 within sensor module 110 can be activated by a user to obtain one or more images of the bone structure. In examples, imaging sensor 172 can be automatically activated computing system 202 without user input. In examples, imaging sensor 172 can be continuously activated to obtain a live video feed of the bone structure. The digital images can be transmitted from sensor module 110 to computing system 202 via wiring 116 or a wireless connection.

At operation 312, a topographical map of the bone structure can be generated and displayed. For example, contour map 230 can be displayed on output device 204, wherein contour map 230 can comprise a representation of a glenoid. The topographical map can comprise highlighted features of the bone structure in order to improve the visibility of such bone structures on output device 204 over what can be readily recognizable in raw video images. Additionally, the topographical map can include features to help visualize depth or height of features on the bone structure. In examples, the topographical map can include contour lines 238.

At operation 314, guidance indicia can be generated and displayed on the topographical map. In examples, the guidance indica can comprise markings, text or instructions for where to place instrumentation on the bone structure. For example, the guidance indica can comprise work area 242, target 244 and no-go zone 246.

At operation 316, instrumentation can be placed on the bone surface using the guidance indicia. For example, instrument 248 can be moved over the bone surface into the view of imaging sensor 172. Thus, surgical personnel can view images of instrument 248 over contour map 230 relative to work area 242 and target 244. As discussed, instrument 248 can comprise a marking device or a pin. The marking device can be used to physically mark the bone at a location for receiving a pin, or the pin can be directly inserted into the bone at the location of target 244. In examples, instrument 248 can be placed via a robotic arm that received coordinate placement instructions from computing system 202 so that instrument 248 can be placed without user intervention.

At operation 318, the camera module can be removed from the retractor. Sensor module 110 can be removed from retractor 101 in order to allow unobstructed access to the interior of retractor 101 and the bone surface therein.

At operation 320, surgical instruments can be placed on the bone structure using the markings or instrumentation placed at operation 316. For example, a pin can be placed at a mark made by the marking device of operation 316 or can comprise a bone modification device, e.g., reamer or broach, disposed over a pin placed at operation 316.

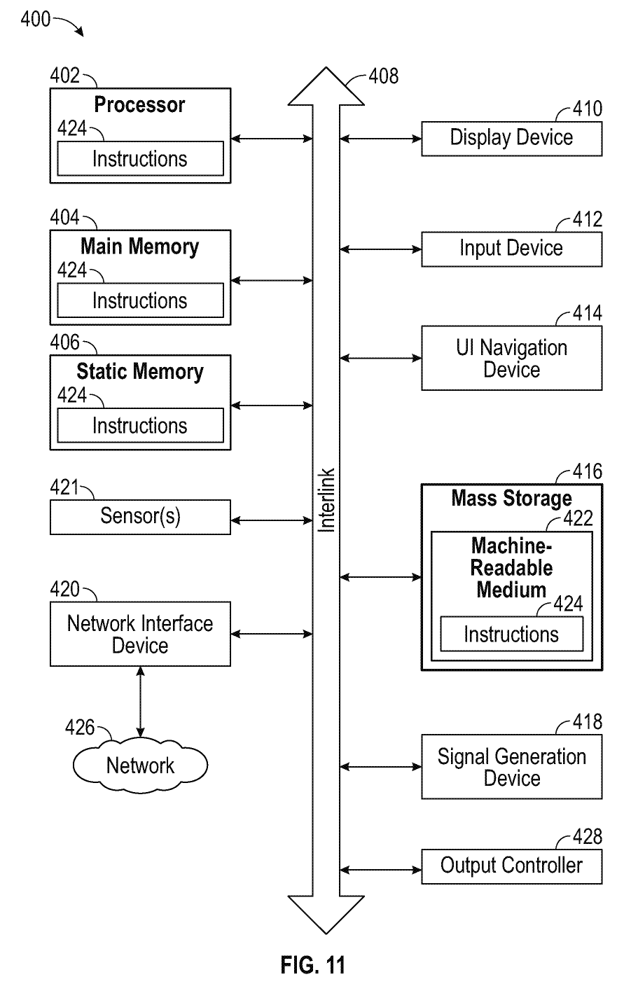

FIG. 11 illustrates a block diagram of an example machine 400 upon which any one or more of the techniques discussed herein may be performed in accordance with some embodiments. For example, machine 400 can comprise computing system 202 of FIG. 7. Machine 400 can comprise an example of a controller for a surgical planning system as described herein. As such instructions 424 can be executed by hardware processor 402 to generate and imaging of anatomical features, identify and/or extrapolate features of the anatomic features, generate a map of the electronic features for display over the imaging, and generate indicia for the placement of surgical instruments on the map.

Machine 400 can operate as a standalone device or may be connected (e.g., networked) to other machines. In a networked deployment, machine 400 may operate in the capacity of a server machine, a client machine, or both in server-client network environments. In an example, machine 400 can act as a peer machine in peer-to-peer (P2P) (or other distributed) network environment. Machine 400 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine 400 can comprise a computer system and can include hardware processor 402 (e.g., a central processing unit (CPU), a graphics processing unit (GPU), a hardware processor core, or any combination thereof), main memory 404 and static memory 406, some or all of which may communicate with each other via interlink 408, which can comprise a bus. Machine 400 may further include display unit 410, alphanumeric input device 412 (e.g., a keyboard), and user interface (UI) navigation device 414 (e.g., a mouse). In an example, display unit 410, alphanumeric input device 412 and UI navigation device 414 may be a touch screen display. Machine 400 may additionally include a drive unit or storage device 416, signal generation device 418 (e.g., a speaker), network interface device 420, and one or more sensors 421, such as a global positioning system (GPS) sensor, compass, accelerometer, or other sensor. Machine 400 may include output controller 428, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

Storage device 416 may include machine readable medium 422 on which is stored one or more sets of data structures or instructions 424 (e.g., software) embodying or utilized by any one or more of the techniques or functions described herein, such as the surgical planning tools, landmark identification and surgical instrument placement guidance indicia, and others. Instructions 424 may also reside, completely or at least partially, within main memory 404, within static memory 406, or within hardware processor 402 during execution thereof by machine 400. In an example, one or any combination of hardware processor 402, main memory 404, static memory 406, or storage device 416 may constitute machine readable media.

While machine readable medium 422 is illustrated as a single medium, the term “machine readable medium” may include a single medium or multiple media (e.g., a centralized or distributed database, and/or associated caches and servers) configured to store the one or more instructions 424. The term “machine readable medium” may include any medium that is capable of storing, encoding, or carrying instructions for execution by machine 400 and that cause machine 400 to perform any one or more of the techniques of the present disclosure, or that is capable of storing, encoding or carrying data structures used by or associated with such instructions. Non-limiting machine readable medium examples may include solid-state memories, and optical and magnetic media.

Instructions 424 may further be transmitted or received over communications network 426 using a transmission medium via network interface device 420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, network interface device 420 may include one or more physical jacks (e.g., Ethernet, coaxial, or phone jacks) or one or more antennas to connect to communications network 426. In an example, network interface device 420 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by machine 400, and includes digital or analog communications signals or other intangible medium to facilitate communication of such software.

Bone map databases, bone feature databases, guidance indicia databases, prosthetic component databases and surgical procedure instruction databases can be stored in main memory 404 and accessed by hardware processor 402. Hardware processor 402 can also receive input (such as at alphanumeric input device 412) relating to patient-specific bone models including patient-specific bone contour information, which can be stored in main memory 404. Machine 400 can execute numerical model simulations of prosthesis fixation and can execute AI/ML engines to evaluate fixation of prostheses in bone. Machine 400 can provide visual indicia of the risks associated with various implant scenarios on display unit 410 (e.g., human interface devices such as output device 204). Machine 400 can be configured to display bone maps, bone features in the bone maps, and guidance for the placement of hardware, such as surgical instruments and prosthetic implants on the bone maps for various surgical procedures, including various arthroplasty procedures, including full and partial shoulder arthroplasty and reverse and anatomic shoulder arthroplasty procedures at display unit 410 to provide real-time feedback to a user when planning and performing the surgical procedure.

The systems, devices and methods discussed in the present application can be useful in performing bone modification procedures by providing intra-operative analysis as to where to place instrumentation during a surgical procedure, such as instrumentation used to guide bone modifying devices to prepare a bone to receive an orthopedic implant.

FIG. 12A is a schematic plan view of expandable hoop retractor 500 of the present disclosure comprising a plurality of linkages pivotably linked together in a collapsed state. FIG. 12B is a schematic plan view of expandable hoop retractor 500 of FIG. 12A in an expanded state. FIGS. 12A and 12B are discussed concurrently.

Expandable hoop retractor 500 can comprise first linkage 502A, second linkage 502B, third linkage 502C, fourth linkage 502D, fifth linkage 502E, sixth linkage 502F, seventh linkage 502G and eighth linkage 502H. The illustrated example discloses eight linkages, however, greater or fewer linkages can be used. Each linkage can be connected to two adjacent linkages by a pivot point 504. One or more of linkages can include a slot 506 that allows another linkage to be slid into the linkage including a slot. Thus, in examples, each of first linkage 502A through eighth linkage 502H can be solid and not include a slot 506. Thus, each of pivot points 504 can comprise a pivot connected between two linkages. However, in examples one or more of pivot points 504 can be positioned in one of slots 506 such that the pinned connection can slide along the length or part of the length of a linkage. Thus, in examples, some of linkages can have different cross-sectional diameters such that one linkage can be slid into another linkage in a telescoping manner. Thus, FIG. 12A shows eighth linkage 502H slid into sixth linkage 502F.

The present disclosure provides several advantageous components over prior art systems and components: an expandable hoop retractor that retracts soft tissue away from the bone surface, a sensor module mountable to the retractor that obtains imaging of the anatomy, a computer system that processes the images to identify anatomical landmarks and generate guidance, multiple embodiments of the expandable hoop retractor, including a design with an arcuate body, hinges, and a latch, and an alternative design with multiple pivoting linkages, a camera assembly with imaging sensors, distance/depth sensors, attachment mechanisms to the retractor, and methods of using the disclosed components and systems in surgical procedures.

The present disclosure provides a plurality of features that provide benefits over prior art systems and methods. In examples, a hoop retractor device can create a hands-free solution for keeping tissue out of the way during shoulder surgery, particularly when accessing the glenoid or another anatomic structure. In examples, an expandable hoop retractor can be locked at different sizes using pins in a configuration that provides a complete 360° portal with one device. In examples, pins can be inserted through the retractor to secure the retractor to bone. The retractors of the present disclosure can save operating room time by being quickly deployed and retaining soft tissue without having to be reset tissue that has slipped off of the retractor. In examples, a camera arm system can attach to the hoop retractor to help with precise implant placement. Systems of the present disclosure can provide real-time imaging of the glenoid surface or another bone surface. Systems of the present disclosure can include software that analyze the bone surface and identify optimal implant placement. Systems of the present disclosure can display guidance on a screen showing where to place pins for implant positioning. In examples, systems of the present disclosure can additionally include depth sensing using, for example, LIDAR capabilities.

### Examples

Example 1 is a system for placing an orthopedic implant, the system comprising: an expandable hoop retractor; and a sensor module mountable to the expandable hoop retractor to obtain imaging of anatomy within the expandable hoop retractor.

In Example 2, the subject matter of Example 1 optionally includes wherein the expandable hoop retractor comprises an annular body having an internal cross-sectional area, wherein the annular body can be selectively locked into discrete expandable positions to vary the internal cross-sectional area.

In Example 3, the subject matter of Example 2 optionally includes wherein the expandable hoop retractor comprises: an arcuate body extending from a first end to a second end; a latch pivotably connected to the arcuate body at the first end; and a first hinge pivotably connected to the arcuate body at the second end; wherein the first hinge and the latch can slidably engage to adjust the internal cross-sectional area.

In Example 4, the subject matter of Example 3 optionally includes a second hinge, wherein the latch can slide between the first hinge and the second hinge.

In Example 5, the subject matter of any one or more of Examples 3–4 optionally include wherein the latch further comprises a slot and the system further comprising a pin insertable through the latch and the first hinge to limit a distance that the latch and the first hinge can slide along each other.

In Example 6, the subject matter of Example 5 optionally includes a coupler connecting the sensor module to the pin.

In Example 7, the subject matter of any one or more of Examples 1–6 optionally include a coupler connecting the sensor module to the expandable hoop retractor.

In Example 8, the subject matter of Example 7 optionally includes wherein the coupler comprises a neck in an adjustable or fixed position, configured to position the sensor module across form an interior of the expandable hoop retractor.

In Example 9, the subject matter of Example 8 optionally includes wherein the coupler comprises a flange that can be inserted into a slot within the expandable hoop retractor.

In Example 10, the subject matter of any one or more of Examples 1–9 optionally include wherein the sensor module comprises an imaging sensor configured to obtain images of an interior of the expandable hoop retractor.

In Example 11, the subject matter of Example 10 optionally includes a controller configured to receive output of the imaging sensor, the controller comprising: memory having instructions stored therein for: generating an image of anatomy within the expandable hoop retractor; identifying anatomic landmarks in the anatomy; and generating a topographical map of the anatomic landmarks relative to the anatomy.

In Example 12, the subject matter of Example 11 optionally includes wherein the sensor module comprises a depth sensor.

In Example 13, the subject matter of Example 12 optionally includes wherein the memory further comprises instructions for generating depth indicia for the topographical map.

In Example 14, the subject matter of any one or more of Examples 11–13 optionally include an output device configured to display: a topographical map of anatomy imaged by the sensor module; and guidance indicia on the topographical map indicating a placement location for a surgical instrument on the anatomy.

In Example 15, the subject matter of Example 14 optionally includes wherein the guidance indicia comprises: an outline surrounding a target area within the anatomy; and a cross-hair for placing a pin in the target area.

Example 16 is a method of preparing a bone for receiving a prosthetic implant, the method comprising: producing a portal in anatomy to access a bone surface; inserting an expandable hoop retractor into the portal to displace soft tissue away from the bone surface; obtaining imaging of the bone surface from a camera attached to the expandable hoop retractor; displaying images of the bone surface on an output device; and displaying guidance indicia on the images in the output device to place a surgical instrument on the bone surface.

In Example 17, the subject matter of Example 16 optionally includes locking the expandable hoop retractor via insertion of a pin into the expandable hoop retractor.

In Example 18, the subject matter of any one or more of Examples 16–17 optionally include wherein inserting the expandable hoop retractor into the portal to displace soft tissue away from the bone surface further comprises expanding an internal cross-sectional area of the expandable hoop retractor to displace soft tissue away from the bone surface.

In Example 19, the subject matter of any one or more of Examples 16–18 optionally include identifying diseased tissue in the bone surface; and displaying depth indicia of the bone surface on the output device.

In Example 20, the subject matter of any one or more of Examples 16–19 optionally include obtaining depth indicia of the bone surface using a distance sensor; and displaying depth indicia of the bone surface on the output device.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The Claimed Invention Is:

1. A system for placing an orthopedic implant, the system comprising:
an expandable hoop retractor; and
a sensor module mountable to the expandable hoop retractor to obtain imaging of anatomy within the expandable hoop retractor.

2. The system of claim 1, wherein the expandable hoop retractor comprises an annular body having an internal cross-sectional area, wherein the annular body can be selectively locked into discrete expandable positions to vary the internal cross-sectional area.

3. The system of claim 2, wherein the expandable hoop retractor comprises:
an arcuate body extending from a first end to a second end;
a latch pivotably connected to the arcuate body at the first end; and
a first hinge pivotably connected to the arcuate body at the second end;
wherein the first hinge and the latch can slidably engage to adjust the internal cross-sectional area.

4. The system of claim 3, further comprising a second hinge, wherein the latch can slide between the first hinge and the second hinge.

5. The system of claim 3, wherein the latch further comprises a slot and the system further comprising a pin insertable through the latch and the first hinge to limit a distance that the latch and the first hinge can slide along each other.

6. The system of claim 5, further comprising a coupler connecting the sensor module to the pin.

7. The system of claim 1, further comprising a coupler connecting the sensor module to the expandable hoop retractor.

8. The system of claim 7, wherein the coupler comprises a neck in an adjustable or fixed position, configured to position the sensor module across form an interior of the expandable hoop retractor.

9. The system of claim 8, wherein the coupler comprises a flange that can be inserted into a slot within the expandable hoop retractor.

10. The system of claim 1, wherein the sensor module comprises an imaging sensor configured to obtain images of an interior of the expandable hoop retractor.

11. The system of claim 10, further comprising:
a controller configured to receive output of the imaging sensor, the controller comprising:
memory having instructions stored therein for:
generating an image of anatomy within the expandable hoop retractor;
identifying anatomic landmarks in the anatomy; and
generating a topographical map of the anatomic landmarks relative to the anatomy.

12. The system of claim 11, wherein the sensor module comprises a depth sensor.

13. The system of claim 12, wherein the memory further comprises instructions for generating depth indicia for the topographical map.

14. The system of claim 11, further comprising an output device configured to display:
a topographical map of anatomy imaged by the sensor module; and
guidance indicia on the topographical map indicating a placement location for a surgical instrument on the anatomy.

15. The system of claim 14, wherein the guidance indicia comprises:
an outline surrounding a target area within the anatomy; and
a cross-hair for placing a pin in the target area.

16. A method of preparing a bone for receiving a prosthetic implant, the method comprising:
producing a portal in anatomy to access a bone surface;
inserting an expandable hoop retractor into the portal to displace soft tissue away from the bone surface;
obtaining imaging of the bone surface from a camera attached to the expandable hoop retractor;
displaying images of the bone surface on an output device; and
displaying guidance indicia on the images in the output device to place a surgical instrument on the bone surface.

17. The method of claim 16, further comprising locking the expandable hoop retractor via insertion of a pin into the expandable hoop retractor.

18. The method of claim 16, wherein inserting the expandable hoop retractor into the portal to displace soft tissue away from the bone surface further comprises expanding an internal cross-sectional area of the expandable hoop retractor to displace soft tissue away from the bone surface.

19. The method of claim 16, further comprising:
identifying diseased tissue in the bone surface; and
displaying depth indicia of the bone surface on the output device.

20. The method of claim 16, further comprising:
obtaining depth indicia of the bone surface using a distance sensor; and
displaying depth indicia of the bone surface on the output device.
